(19) United States
(12) Patent Application Publication
(10) Pub. No.: US 2011/0105865 A1
(43) Pub. Date: May 5, 2011

(54) DIFFUSE REFLECTANCE SPECTROSCOPY DEVICE FOR QUANTIFYING TISSUE ABSORPTION AND SCATTERING

(75) Inventors: Bing Yu, Cary, NC (US); Nimala Ramanujan, Chapel Hill, NC (US); Justin Y. Lo, Durham, NC (US)

(73) Assignee: DUKE UNIVERSITY, Durham, NC (US)

(21) Appl. No.: 12/989,595

(22) PCT Filed: Apr. 24, 2009
(86) PCT No.: PCT/US2009/041732

§ 371 Date: Dec. 3, 2010

Related U.S. Application Data
(60) Provisional application No. 61/047,602, filed on Apr. 24, 2008.

(51) Int. Cl. A61B 5/1455

(52) U.S. Cl. 600/310

(57) ABSTRACT

A diffuse reflectance spectroscopy system for quantifying electromagnetic absorption and scattering in a tissue is provided. Also provided are optical probes and methods for imaging a tissue mass. In some embodiments, the methods include the steps of contacting a tissue mass with an optical probe, wherein the optical probe includes at least one entity for emitting light that interacts with a tissue mass and then is remitted to a collecting entity, for collecting the light that has interacted with the tissue mass, wherein the collecting entity comprises a detector comprising one or more photodiodes; measuring turbid spectral data of the tissue mass using the optical probe; converting the turbid spectral data to at least one of absorption and scattering spectral data via a Monte Carlo algorithm or a diffusion algorithm; and quantifying tissue compositions and scatterer size in a tissue mass using the at least one of absorption and scattering spectral data.

### RELATED APPLICATIONS

The presently disclosed subject matter claims the benefit of U.S. Provisional Patent Application Ser. No. 61/047,602, filed Apr. 24, 2008, the disclosure of which is incorporated herein by reference in its entirety.

### GOVERNMENT INTEREST

This presently disclosed subject matter was made with U.S. Government support under an Era of Hope Scholar award awarded by U.S. Department of Defense Breast Cancer Research Program DOD BCRP). Thus, the U.S. Government has certain rights in the presently disclosed subject matter.

### TECHNICAL FIELD

The presently disclosed subject matter relates to devices and systems for quantifying tissue absorption and scattering using diffuse reflectance spectroscopy. The presently disclosed subject matter also relates to methods for employing the disclosed devices and systems for imaging a tissue mass.

### BACKGROUND

UV-visible diffuse reflectance spectroscopy (UV-VIS DRS) is sensitive to the absorption and scattering properties of biological molecules in tissue and thus can be used as a tool for quantitative tissue physiology in vivo. A major absorber of light in mucosal tissue in the visible range is hemoglobin (Hb), which shows distinctive, wavelength-dependent absorbance characteristics depending on its concentration and oxygenation. Tissue scattering is sensitive to the size and density of cellular structures such as nuclei and mitochondria. Thus, DRS of tissues can quantify changes in oxygenation, blood volume, and alterations in cellular density and morphology. Some potential clinical applications of UV-VIS DRS include monitoring of tissue oxygenation (Bigio & Bown, 2004), precancer and cancer detection (Zonios et al., 1999; Mirabal et al., 2002) intraoperative tumor margin assessment (Lin et al., 2001) and assessing tumor response to cancer therapy (Bigio & Bown, 2004).

A fiber optic DRS system (Zhu et al., 2005) and a fast inverse Monte Carlo (MC) model of reflectance (Palmer & Ramanujam, 2006a) have been developed to nondestructively and rapidly quantify tissue absorption and scattering properties. The system included a 450-W xenon lamp, a monochromator, a fiber optic probe, an imaging spectrograph, and a CCD camera. This technology has been shown to be capable of quantifying breast tissue physiological and morphological properties, and that these quantities can be used to discern between malignant and non-malignant tissues with sensitivities and specificities exceeding 80% (Zhu et al, 2006).

A simpler, low cost, portable reflectance spectrometer, capable of making fast measurements and easily extendable into a spectral imaging platform for mapping tissue optical properties is desirable for clinical applications including, but not limited to intraoperative assessment of tumor margins. Previous studies have attempted to develop a portable DRS probe for cancer detection. Cerussi et al. 2006 describes a handheld (5×8×10 cm) laser breast scanner (LBS) based on frequency-domain near-infrared spectroscopy for breast cancer detection. The LBS probe consists of a fiber bundle for illumination and an avalanche photodiode module placed 22 mm from the fiber bundle for detection. Feather et al. 1988 reported a portable diffuse reflectometer that uses nine LEDs at three visible wavelengths to illuminate skin and a photodiode to collect diffusely reflected light through a 7-mm aperture. The LBS has a sensing depth over 1 cm, but is difficult to multiplex into a spectral imaging device because of the size of the device. The LED-photodiode-based reflectometer is extendable to imaging, but measurements based on this device do not provide quantitative endpoints such as absorption and scattering that relate to the underlying biology of the tissue.

What is needed, then, is a low cost, portable reflectance spectrometer, capable of making fast measurements and easily extendable into a spectral imaging platform for mapping tissue optical properties.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides diffuse reflectance spectroscopy systems for quantifying light absorption and scattering in a tissue mass. In some embodiments, the systems comprise an optical probe comprising at least one entity for emitting light that interacts with a tissue mass and then is remitted into a collecting entity, wherein the collecting entity comprises a detector comprising one or more photodiodes; and a processing unit for converting collected light, via a Monte Carlo algorithm or a diffusion algorithm into absorption and scattering data. In some embodiments, the entity for emitting light is present at a fixed distance external to a photodiode. In some embodiments, the entity for emitting light comprises one or more illumination fibers, each illumination fiber being present at a fixed distance external to a photodiode, optionally adjacent to a photodiode. In some embodiments, the entity for emitting light comprises one or more illumination fibers, each illumination fiber being present within a photodiode. In some embodiments, the illumination fiber is disposed longitudinally along the center of the photodiode. In some embodiments, the photodiode comprises an aperture, and the illumination fiber is disposed within the aperture, optionally wherein spacing is present to vary the distance between the center of the aperture and/or fiber and an edge of the photodiode.

In some embodiments, the diffuse reflectance spectroscopy systems of the presently disclosed subject matter further comprise a light source coupled to the entity for emitting light, wherein the light source optionally comprises a lamp or a plurality of light-emitting diodes (LEDs). In some embodiments, the lamp or each LED emits light at one or more wavelengths between about 400 nm and about 950 nm.

In some embodiments, the diffuse reflectance spectroscopy system of the presently disclosed subject matter further comprise a dispersing element such as a monochromator or a filter wheel operably attached to the system between the light source and entity for emitting light.

In some embodiments, the diffuse reflectance spectroscopy systems of the presently disclosed subject matter further comprise a monochromator or a filter wheel attached to the light source. In some embodiments, the entity for emitting light and collecting entities are encased in a housing, where the entity for emitting light is at a proximal end of the housing and the one or more photodiodes are at a distal end of the housing, the one or more photodiodes each comprising an aperture, whereby the entity for emitting light provides backlit illumination through each aperture into one or more photodiodes. In some embodiments, the housing comprises one or more reflective interior surfaces.

In some embodiments of the presently disclosed subject matter, the one or more photodiodes comprises an array of photodiodes. In some embodiments, the array is present in a configuration selected from a group consisting of a square, a rectangular, and a circular configuration. In some embodiments, the Monte Carlo algorithm includes an inverse Monte Carlo reflectance algorithm, a scaled Monte Carlo reflectance algorithm, or a combination thereof.

The presently disclosed subject matter also provides optical probes. In some embodiments, the optical probes comprise at least one entity for emitting light into a tissue mass and at least one collecting entity for collecting light that has interacted with a tissue mass, wherein the collecting entity comprises one or more photodiodes. In some embodiments, the entity for emitting light is present at a fixed distance external to a photodiode. In some embodiments, the entity for emitting light comprises one or more illumination fibers, each illumination fiber being present at a fixed distance external to a photodiode. In some embodiments, the entity for emitting light comprises one or more LEDs. In some embodiments, each LED emits light at a wavelength between about 400 nm and about 950 nm. In some embodiments, the optical probe further comprises a housing, and the entity for emitting light is at a proximal end of the housing and the one or more photodiodes are at a distal end of the housing, whereby the entity for emitting light provides backlit electromagnetic radiation with respect to the one or more photodiodes. In some embodiments, the housing comprises one or more reflective interior surfaces. In some embodiments, the optical probes of the presently disclosed subject matter comprise one or more illumination fibers, each illumination fiber being present within a photodiode. In some embodiments, the illumination fiber is disposed longitudinally along the center of the photodiode. In some embodiments, the optical probes of the presently disclosed subject matter comprise a buffer between the photodiode and the illumination fiber. In some embodiments, the one or more photodiodes comprises an array of photodiodes. In some embodiments, the array is present in a configuration selected from a group consisting of a square, a rectangular, and a circular configuration. In some embodiments, the entity for emitting light comprises a light source. In some embodiments, the light source further comprises a monochromator or a filter wheel.

The presently disclosed subject matter also provides methods for imaging a tissue mass. In some embodiments, the methods comprise contacting a tissue mass with an optical probe, wherein the optical probe comprises at least one entity for emitting light that interacts with a tissue mass and then is remitted to a collecting entity, for collecting the light that has interacted with the tissue mass, wherein the collecting entity comprises a detector comprising one or more photodiodes; measuring turbid spectral data of the tissue mass using the optical probe; converting the turbid spectral data to at least one of absorption and scattering spectral data via a Monte Carlo algorithm or a diffusion algorithm; and quantifying tissue compositions and scatterer size in a tissue mass using the at least one of absorption and scattering spectral data. In some embodiments, the entity for emitting light is present at a fixed distance external to a photodiode. In some embodiments, the entity for emitting light comprises one or more illumination fibers, each illumination fiber being present at a fixed distance external to a photodiode. In some embodiments, a distal end of each of the one or more illumination fibers is substantially coplanar with a collecting surface of each of the one of more photodiodes. In some embodiments, each illumination fiber is present within a photodiode. In some embodiments, the illumination fiber is disposed longitudinally along the center of the photodiode. In some embodiments, the presently disclosed methods employ the optical probes that comprise a buffer between the photodiode and the illumination fiber. In some embodiments, the emitting entity of the optical probe comprises a lamp or a plurality of LEDs. In some embodiments, each lamp or LED emits light at one or wavelength between about 400 nm and about 950 nm.

In some embodiments, the presently disclosed methods employ optical probes that further comprise a housing, and the entity for emitting light is at a proximal end of the housing and the one or more photodiodes are at a distal end of the housing, whereby the entity for emitting light provides backlit electromagnetic radiation (through a hole or transparent window at the center of a photodiode) with respect to the one or more photodiodes. In some embodiments, the housing of optical probe comprises one or more reflective interior surfaces. In some embodiments of the presently disclosed methods, the one or more photodiodes comprises an array of photodiodes. In some embodiments, the array is present in a configuration selected from a group consisting of a square, a rectangular, and a circular configuration. In some embodiments, the optical probe is operably attached to a light source. In some embodiments, the methods of the presently disclosed subject matter further comprise employing a monochromator or a filter wheel operably attached to the system between the light source and the optical probe. In some embodiments, the turbid spectral data comprises diffuse reflectance spectral data of the tissue mass. In some embodiments, the Monte Carlo algorithm includes an inverse Monte Carlo reflectance algorithm, a scaled Monte Carlo reflectance algorithm, or a combination thereof.

It is an object of the presently disclosed subject matter to provide a diffuse reflectance spectroscopy and/or or spectral imaging system for quantifying electromagnetic absorption and scattering in a tissue mass, and to provide related components and methods.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a block diagram of an optical spectrometer system for determining biomarker concentrations in a tissue mass according to an embodiment of the subject matter described herein;

FIG. 2A is a schematic block diagram of a system 200 in accordance with the presently disclosed subject matter;

FIGS. 2B-2D are schematic end views of embodiments of an optical probe 202 in accordance with the presently disclosed subject matter;

FIG. 3A is a schematic block diagram of an embodiment 300 of a system of the presently disclosed subject matter;

FIGS. 3B and 3C are schematic sectional views of embodiments of optical probe 302 of the presently disclosed subject matter; and

FIG. 4 is a block diagram flow chart of a process in accordance with the presently disclosed subject matter.

FIG. 5 is a schematic block diagram of an embodiment 500 of an optical probe array of the presently disclosed subject matter.

FIG. 6 is a plot of calibrated measured and MC-fitted tissue phantom spectra. Circles represent for the calibrated measured data points and the line represents the calibrated MC-fitted data plot.

FIGS. 7A and 7B are plots of extracted versus expected absorption coefficient (FIG. 7A) and reduced scattering coefficient (FIG. 7B). The line represents perfect agreement between the two data sets, and the larger circles and smaller circles represent the system of FIG. 1 and a system of the presently disclosed subject matter, respectively.

FIGS. 8A and 8B are plots of a comparison of μₐ and μₛ′ extractions by the system of FIG. 1 and a system of the presently disclosed subject matter, respectively. The line represents perfect agreement between the two data sets, and the gray circles and black circles represent the system of FIG. 1 and a system of the presently disclosed subject matter, respectively.

FIG. 9 is a plot of experimental reflectance spectra from lightest and darkest phantoms with five wavelengths chosen to for MC inversions. The lines represent measured spectra and the circles represent simulated LED λ.

FIGS. 10A and 10B are plots of extractions of μₐ and μₛ′, respectively, after wavelength reduction simulation. The lines represent the perfect fit and the circles of the λ-reduced extractions.

FIGS. 11A and 11B are plots of reconstructed hemoglobin (Hb) spectra averaged over all phantoms using extracted μₐ values at five chosen wavelengths, and extractions of Hb concentration by inverting wavelength-reduced data, respectively.

### DETAILED DESCRIPTION

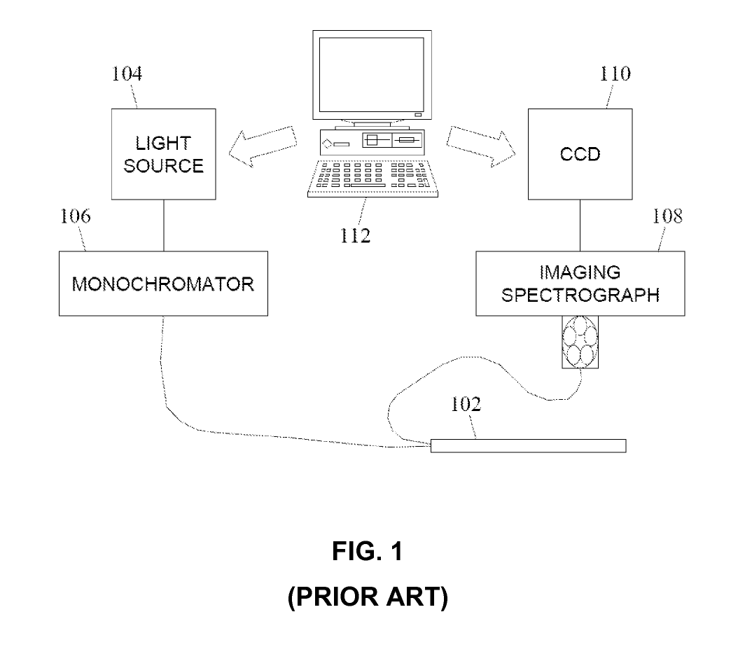

Referring now to the Figures, FIG. 1 depicts an exemplary prior art optical spectrometer system 100 that includes a fiber optic probe 102. Spectrometer system 100 may also include a light source 104 (e.g., a xenon lamp), a monochromator 106 (e.g., a scanning double-excitation monochromator), an imaging spectrograph 108, a charged-couple device (CCD) unit 110, and a processing unit 112 (e.g., a computer).

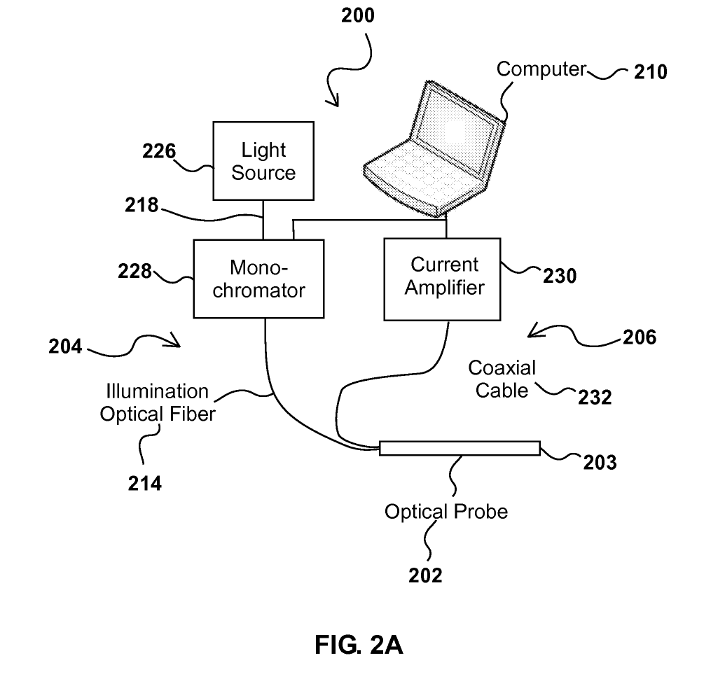

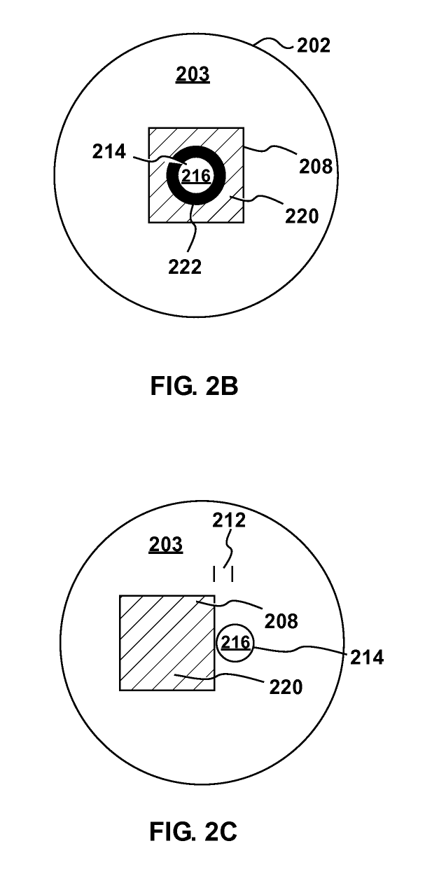

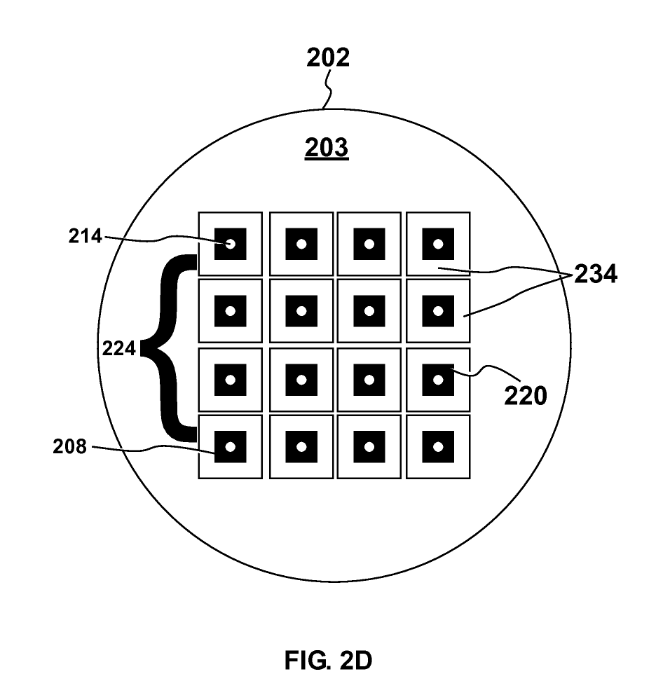

Referring now to FIGS. 2A-2D, an exemplary diffuse reflectance spectroscopy system for quantifying electromagnetic absorption and scattering in a tissue mass of the presently disclosed subject matter is presented generally at 200. System 200 comprises an optical probe 202 having a tip 203 comprising at least one emitting entity 204 for emitting electromagnetic radiation (such as but not limited to light) into a tissue mass and at least one collecting entity 206 for collecting electromagnetic radiation that has interacted with the tissue mass. Collecting entity 206 can comprise a detector, such as but not limited to one or more photodiodes 208. System 200 comprises processing unit 210 (such as but not limited to a computer) for converting collected electromagnetic radiation to at least one of absorption and scattering data, via a Monte Carlo algorithm or a diffusion algorithm and quantifying absorption and scattering in the tissue mass using the absorption and scattering data. The Monte Carlo algorithm can include an inverse Monte Carlo reflectance algorithm, a scaled Monte Carlo reflectance algorithm, or a combination thereof.

Continuing with reference to FIGS. 2A-2D, and with particular reference to FIG. 2B, in some embodiments emitting entity 204 can comprise one or more illumination fibers 214, wherein each illumination fiber 214 is present within each photodiode 208. Optionally, illumination fiber 214 is disposed longitudinally along the center of photodiode 208 present at tip 203. Further optionally, photodiode 208 can comprise an aperture 222. Illumination fiber 214 is disposed within aperture 222, optionally wherein spacing is present to vary a distance between the center of aperture 222 and/or fiber 214 and an edge 209 of photodiode 208. Varying this distance can tune the sensing depth.

Continuing with reference to FIGS. 2A-2D, and with particular reference to FIG. 2C, emitting entity 204 can comprise one or more illumination optical fibers 214. In some embodiments, such as that shown in FIG. 2C, each illumination fiber can be present at a fixed distance 212 external to photodiode 208, optionally adjacent to photodiode 208. Distal end 216 of each of the one or more illumination fibers 214 can be substantially coplanar with a collecting surface 220 at the tip 203 of each of the one of more photodiodes 208. In some embodiments, there is one fiber 214 for each photodiode 208.

Continuing with reference to FIGS. 2A-2D, and with particular reference to FIG. 2D, system 200 can comprise comprises an array 224 of photodiodes 208. Array 224 can be present in a configuration selected from the group including but not limited to square, rectangular, and circular. Any suitable number of photodiodes 208 can be included in array 224. By way of non-limiting example, array 224 can be present in a 2×2, a 3×3, a 4×4, and/or a 5×5 configuration. Indeed, array 224 can comprise as many as a hundred pixels if desired. Array 224 can be mounted on a support 234.

Continuing with reference to FIGS. 2A-2D, emitting entity 204 can comprise light source 226, wherein light source 226 is coupled to illumination fiber 214. Light source 226 optionally comprises a lamp, such as but not limited to a Xenon (Xe) lamp. Light source 226 can emit light at a wavelength between about 400 nm and about 950 nm, include but not limited to 405 nm, 430 nm, 450 nm, 470 nm, 505 nm, 530 nm, 570 nm, and/or 590 nm. Emitting entity 204 can comprise a monochromator 228 operably attached in system 200 between light source 226 and optical probe 202 via the one or more illumination fibers 214. Collecting entity 206 can comprise a current amplifier 230 operably connected to one or more photodiodes 210 by coaxial cable 232, and further operably connected to processor 210.

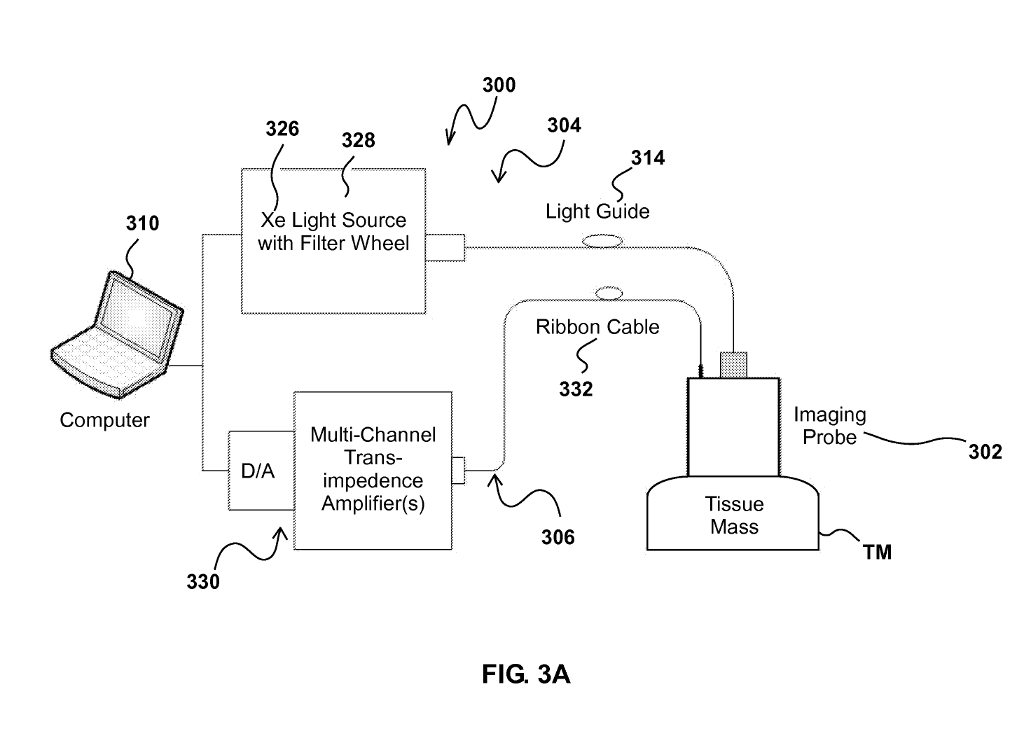

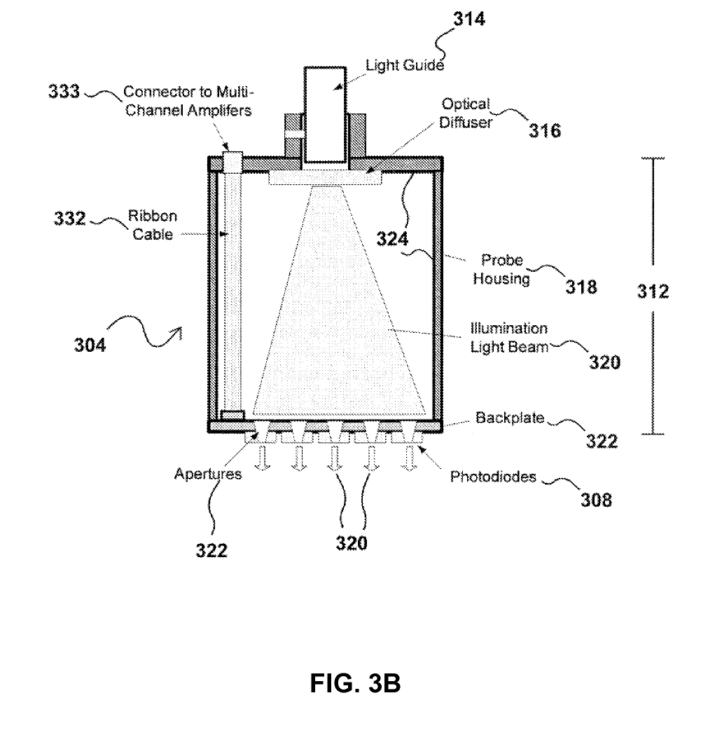

Referring now to FIGS. 3A-3C, another exemplary embodiment of a diffuse reflectance spectroscopy system for quantifying electromagnetic absorption and scattering in a tissue mass is presented generally at 300. System 300 comprises an optical probe 302 comprising at least one emitting entity 304 for emitting electromagnetic radiation (such as but not limited to light) into a tissue mass TM and at least one collecting entity 306 for collecting electromagnetic radiation that has interacted with tissue mass TM. Collecting entity 306 can comprise a detector, such as but not limited to one or more photodiodes 308. System 300 comprises processing unit 310 (such as but not limited to a computer) for converting collected electromagnetic radiation to at least one of absorption and scattering data, via a Monte Carlo algorithm or a diffusion algorithm and quantifying absorption and scattering in the tissue mass using the absorption and scattering data. The Monte Carlo algorithm can include an inverse Monte Carlo reflectance algorithm, a scaled Monte Carlo reflectance algorithm, or a combination thereof.

Continuing with reference to FIGS. 3A-3C, emitting entity 304 can provide direct illumination via a light source 326, such as a lamp, such as but not limited to a Xenon (Xe) lamp, or a plurality of light-emitting diodes (LEDs; shown at 336 in FIG. 3C), a plurality of laser diodes, or a combination thereof. Thus, back illumination can be provided for spectral imaging. Light source 326 can emit light at a wavelength between about 400 nm and about 950 nm, include but not limited to 405 nm, 430 nm, 450 nm, 470 nm, 505 nm, 530 nm, 570 nm, and/or 590 nm. With regard to LEDs 336, these can be arranged in any pattern, and single and/or multiple LED can be present for each color. Filter wheel 328 can be operably connected to light source 326. Emitting entity 304 can comprise a light guide 314 connecting light source 326 to optical probe 302.

Continuing with reference to FIGS. 3A-3B, optical probe 302 further comprises a housing 318. Light guide 314 and optical diffuser 316 (which is optional in housing 318), which comprise parts of emitting entity 304, are at a proximal end of housing 318 and one or more photodiodes 308 are at a distal end of housing 318. Fixed distance 312 is defined between proximal and distal ends of housing 318. Fixed distance 312 can be adjustable to any desired distance. The one or more photodiodes 308 each comprise an aperture 322. Light guide 314 provides backlit electromagnetic radiation 320 through each aperture 322 in the one or more photodiodes 308. Optionally, apertures 322 can comprise a transparent window. Photodiodes 308 can be mounted on backplate 323. Housing 318 can comprise one or more reflective interior surfaces 324. Collecting entity 306 can comprise a multi-channel trans-impedance amplifier 330 operably connected to one or more photodiodes 308 by ribbon cable 332 and connector 333, and further operably connected to processor 310. Alternatively or in addition, multi-channel amplifier 330 can be directly mounted on backplate 323 or on a PCB board plugged into backplate 323.

Continuing with reference to FIGS. 3A and 3C, emitting entity 304 comprises optical probe 302 having an alternative housing 318′. LEDs 336 are mounted at a proximal end of housing 318′ on a PCB 334 with a heat sink and reflective inner surface 335. One or more photodiodes 308 are at a distal end of housing 318′. Fixed distance 312′ is defined between proximal and distal ends of housing 318′. Fixed distance 312′ can be adjustable to any desired distance. The one or more photodiodes 308 each comprise an aperture 322. LEDs 336 provide backlit electromagnetic radiation 338, which can be of varying wavelengths, through each aperture 322 in the one or more photodiodes 308. Optionally, apertures 322 can comprise a transparent window. Photodiodes 308 can be mounted on backplate 323′, which has a reflective internal surface 337. Housing 318′ can comprise one or more reflective interior surfaces 324′. Collecting entity 306 can comprise a multi-channel trans-impedance amplifier 330 operably connected to one or more photodiodes 308 by cable 332′ and further operably connected to processor 310. Alternatively or in addition, multi-channel amplifier 330 can be directly mounted on backplate 323 or on a PCB board plugged into backplate 323.

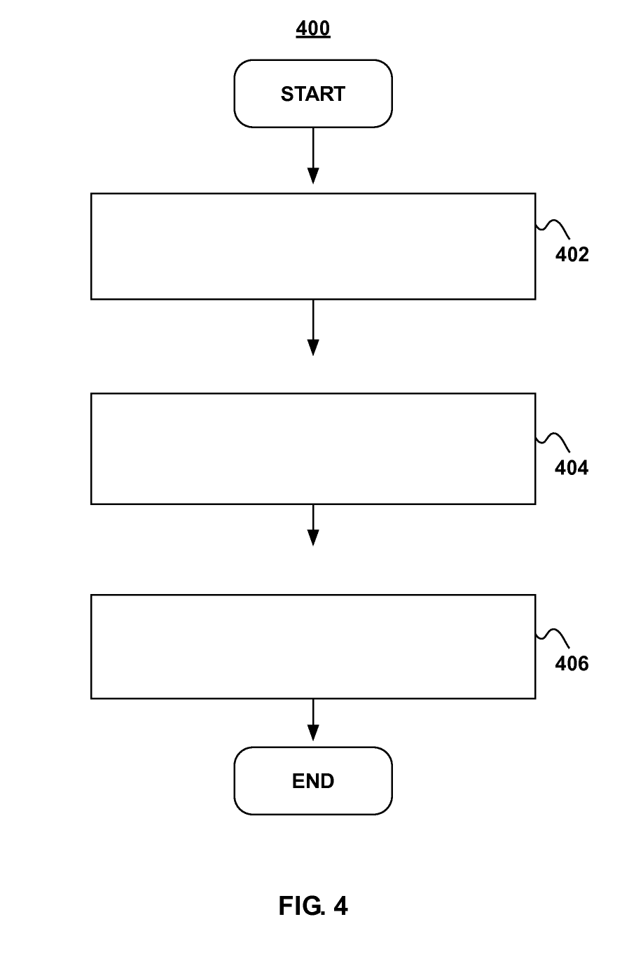

In some embodiments, system 200 or 300 can be employed in accordance with the following representative methods. Indeed, with reference to FIG. 4, in some embodiments, a method 400 for imaging a tissue mass is provided. In block 402, a tissue mass is contacted with an optical probe 202 or 302, wherein optical probe 202, 302 comprises at least one emitting entity 204, 304 for emitting electromagnetic radiation into a tissue mass TM and at least one collecting entity 206, 306 for collecting the electromagnetic radiation that has interacted with the tissue mass, wherein the collecting entity 206, 306 comprises one or more photodiodes 208, 308. In block 404, turbid spectral data of the tissue mass TM is measured using optical probe 202, 302. In block 406 the turbid spectral data is converted to at least one of absorption and scattering spectral data via a Monte Carlo algorithm or a diffusion algorithm; and quantifying tissue compositions and scatterer size in a tissue mass using the at least one of absorption and scattering spectral data. The turbid spectral data can comprise diffuse reflectance spectral data of the tissue mass. The Monte Carlo algorithm can include an inverse Monte Carlo reflectance algorithm, a scaled Monte Carlo reflectance algorithm, or a combination thereof.

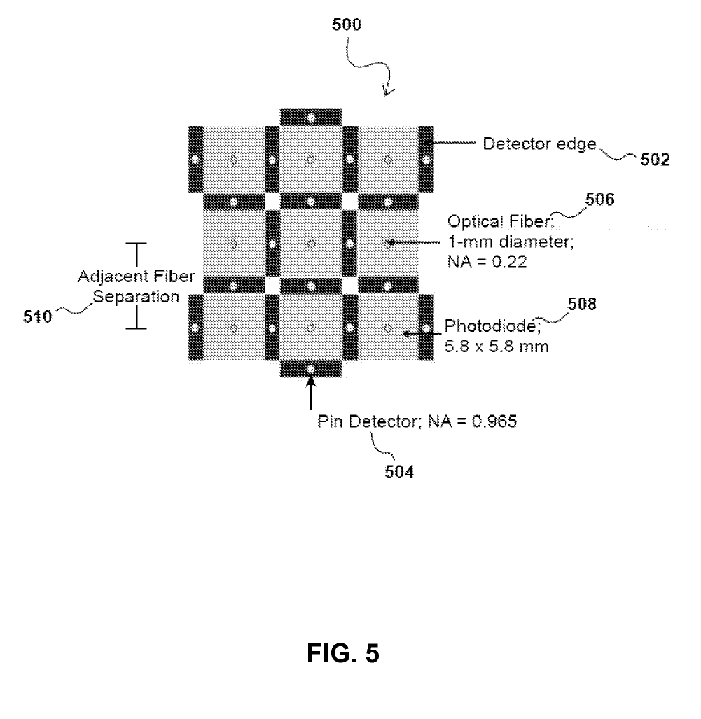

Referring now to FIG. 5, an exemplary embodiment of an optical probe array for use in a diffuse reflectance spectroscopy system of the presently disclosed subject matter is presented generally at 500. Array 500 comprises nine photodiodes 508 (in some embodiments, 5.8×5.8 mm Si photodiodes), each photodiode 508 being adjacent to at least one detector edge 502. Each detector edge 502 can comprise a pin detector 504 (in some embodiments, a pin Si detector that has a numerical aperture (NA) of 0.965). Each photodiode 508 also can have present within it an optical fiber 506 (in some embodiments, a 1-mm diameter optical fiber illumination fiber with an NA of 0.22) such that there is an adjacent fiber separation 510 (in some embodiments, an adjacent fiber separation of 8.48 mm) between the center of one optical fiber 506 to the center of an adjacent optical fiber 506.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### System Modification and Probe Geometry

A schematic representation of a benchtop system is shown in FIG. 1. The system included a 450 W Xenon Arc lamp (J Y Horiba, Edison, N.J., United States of America) and a scanning monochromator (Gemini 180; J Y Horiba) as the source. A fiber optic probe with a core of 19 illumination fibers surrounded by a ring of 18 detection fibers was used for illumination and collection. The individual illumination and collection fibers had a diameter of 200 μm and a numerical aperture (NA) of 0.22. The effective illumination diameter of the probe was 1 mm. The remitted light was collected by the outer ring of detection fibers and coupled through an imaging spectrograph (Triax 320; J Y Horiba) and detected by a CCD (Symphony; J Y Horiba). The system specifications were described in greater detail in Zhu et al., 2005. The system generally corresponds to that described in FIG. 1 hereinabove.

Exemplary Embodiment A. In one embodiment, the hybrid system of the presently disclosed subject matter shown in FIG. 2A included a 450-W xenon lamp and monochromator (J Y Horiba, Edison, N.J., United States of America), a 1-mm illumination optical fiber (numerical aperture (NA)=0.22), a 2.4-mm silicon photodiode (S1226, Hamamatsu, Japan) with a low-noise current amplifier (PDA-750, Terahertz Technologies Inc., Oriskany, N.Y., United States of America), and a laptop computer. The hybrid system used the same light source and monochromator and an illumination fiber with similar diameter and NA as the original system. A difference between the original system and the hybrid system disclosed herein was that the photodiode and current amplifier in the new system replaced the collection fibers, spectrograph, and CCD camera employed in the original system.

At the distal end of the probe depicted in FIG. 2C, the edge of the photodiode was trimmed to the active area and transparent epoxy was used to bond the cleaved fiber adjacent to the photodiode, such that the center-to-center distance between the fiber and the photodiode was 2.1 mm. The overall diameter of the probe tip was 6 mm. The maximum power out of the illumination fiber was 130 μW at 470 nm, and the minimum power was 65 μW at 590 nm. This system had significantly lower cost and better collection efficiency than the original system because of the larger NA of the silicon photodiode (NA=0.96) and its direct contact with the tissue mass. It can also be easily multiplexed into a spectral imaging device by interfacing a bundle of optical fibers to the exit slit of the monochromator and separating the fibers at the distal end, such that each fiber is coupled to a discrete photodiode within a large matrix of photodiodes.

Exemplary Embodiment B. In another embodiment of the hybrid system of the presently disclosed subject matter, the imaging spectrograph and CCD were replaced with a 5.8×5.8 mm silicone photodiode (S1227-66BR; Hamamatsu USA). To minimize the separation between illumination and detection areas and to maximize the collection efficiency, a hole with a diameter of 1.3 mm was drilled in the center of the photodiode. The careful drilling of the photodiode minimized mechanical damage and ensured similar detection performance. The only difference between the drilled and un-drilled photodiode was the total area of detection, which is 32.51 mm² for the drilled detector vs. 33.64 mm² for the un-drilled detector (the ratio of the areas is 0.97). The ratio of the signals detected by the drilled and undrilled detectors when exposed to an incandescent bulb was 0.96, which is similar to the loss of detection area of the drilled detector vis-à-vis the undrilled detector.

A single optical fiber with a core diameter of 1 mm and numerical aperture of 0.22 was fitted through the hole to illuminate the sample. Schematics of the system and probe tip are illustrated in FIGS. 2A and 2B, respectively. This illumination and collection geometry was similar to that of the fiber optic probe geometry shown in FIG. 1. The photodiode was connected to a photodiode amplifier (PDA-750; Terahertz Technologies Inc., Oriskany, N.Y., United States of America) via a coaxial cable for diffuse reflectance measurements. The performance metrics of the original benchtop system and the modified system were also compared.

### Example 2

### Optical Measurements of Synthetic Tissue Phantoms

Exemplary Embodiment A. To evaluate the performance of the modified system of the presently disclosed subject matter shown in FIG. 2A, a series of experiments were conducted on homogeneous tissue phantoms. Prior to the phantom experiments, the long-term drift and signal-to-noise ratio (SNR) of the system were characterized. It was determined that the drift of the system was less than 1 nA over 2 hours with the lamp on and the probe tip in contact with the surface of a liquid phantom. By taking three consecutive diffuse reflectance (DR) spectra from 400 to 600 nm in the darkest phantom among the 10 phantoms described hereinbelow, an average SNR [=20 log(mean intensity/standard deviation)] of 42.9 dB over all wavelengths and a minimum SNR of 24.6 dB at 410 nm, which is close to the Soret band of oxy-Hb, were calculated.

Phantoms with absorption coefficient (μₐ) and reduced scattering coefficient (μₛ) representative of human breast tissues in the 400 to 600-nm wavelength range (see Palmer & Ramanujam, 2006a; U.S. Patent Application Publication Nos. 2007/0232932 and 2008/027009) were created with the scatterer (1-μm diameter polystyrene spheres; 07310-15, Polysciences, Inc., Warrington, Pa., United States of America) and variable concentrations of the absorber (hemoglobin; H0267, Sigma-Aldrich Co., St. Louis, Mo., United States of America). Two sets of liquid phantoms were created by titrating the absorber at two scattering levels, and all DR measurements were made the day the phantoms were prepared.

The first set of phantoms (1A to 1E) included five low-scattering phantoms (wavelength-averaged μₛ′ was about 10.6 cm⁻¹) with wavelength-averaged μₐ of 0.49, 0.88, 1.28, 1.58, and 1.97 cm⁻¹ over the 400 to 600-nm range. The second set (2A to 2E) included five high-scattering phantoms (wavelength-averaged μₛ′ was about 18.5 cm⁻¹) with the same μₐ values as the first set. A complete DR spectrum was collected from each phantom by scanning the bandpass of the monochromator (4.5 nm) from 400 to 600 nm at increments of 5 nm. A DR spectrum was also obtained from a SPECTRALON® 99% diffuse reflectance puck (SRS-99-010, Labsphere, Inc., North Sutton, N.H., United States of America) with the probe in contact with the puck immediately after the phantom measurements with the same instrument settings.

An inverse MC model (see Palmer & Ramanujam, 2006a) was used to extract the μₐ and μₛ′ of the liquid phantoms. The model was validated in both phantom and clinical studies (see Palmer & Ramanujam, 2006a; Zhu et al., 2006). The MC forward model assumed a set of absorbers (oxy-Hb with known extinction coefficients measured using a spectrophotometer in this case) were present in the medium. The scatterer (polystyrene microsphere in this study) was assumed to be single-sized, spherically shaped, and uniformly distributed. The μₐ(λ) of the medium were calculated from the concentration of each absorber and the corresponding extinction coefficients using Beers' law. The μₛ′(λ) and anisotropy factor were calculated using Mie theory (Bohren & Huffman, 1983; Huffman, 1998; see also U.S. Patent Application Publication Nos. 2007/0232932 and 2008/0270091). The μₐ(λ) and μₛ′(λ) were then input into a scalable MC model of light transport to obtain a modeled DR spectrum. In the inverse model, the modeled DR was adaptively fitted to the measured tissue DR. When the sum of square error between the modeled and measured DR was minimized, the concentrations of absorber, from which μₛ can be derived, and μₛ′ were extracted.

To experimentally compare measured phantom spectra to MC simulated phantom spectra for the fitting process, the “calibrated” DR spectrum of the target phantom for which the optical properties were quantified, was divided point by point by the “calibrated” DR spectrum of a reference phantom with known optical properties. The term “calibrated” in both cases refers to the normalization of the DR spectrum to that measured from the SPECTRALON® puck for correction of the wavelength-dependent response of the instrument. In the instant phantom study, phantom 1C (wavelength-averaged μₐ=1.28 cm⁻¹, wavelength-averaged μₛ′=10.6 cm⁻¹) was selected as a reference phantom and the remaining nine phantoms were used as targets.

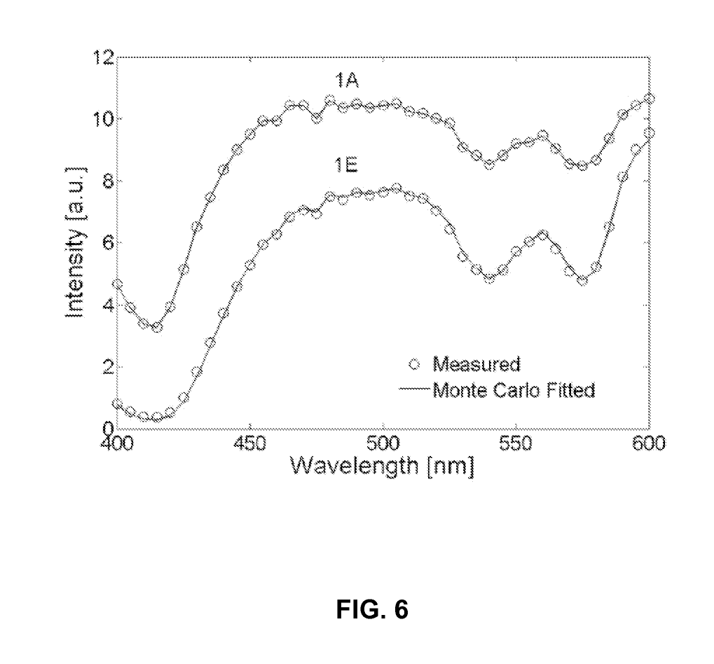

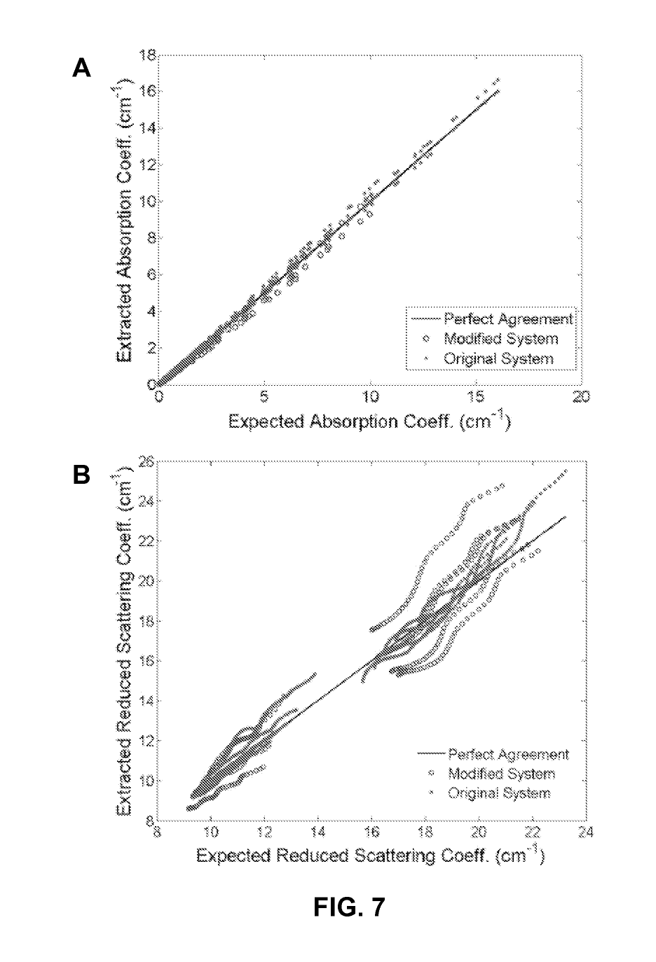

FIG. 6 shows the SPECTRALON® puck-calibrated reflectance spectra for two phantoms, 1A and 1E, and the corresponding fits to the MC model. The three valleys at 415, 540, and 575 nm on the spectra for both phantoms corresponded to the Soret (400 to 450 nm), α (540 nm), and β (569 nm) bands of oxygenated Hb, respectively. There was excellent agreement between the measured spectra and the fits. FIGS. 7A and 7B show the extracted versus expected μₐ and μₛ′ for all wavelengths over the 400 to 600-nm range quantified with the modified and original systems for the similar range of optical properties. The 10 phantoms tested with the modified system had an overall μₐ range of 0.035 to 10 cm⁻¹ and a μₛ′ range of 9.2 to 22.2 cm⁻¹, while that tested with the original system had overall μₐ and μₛ′ ranges of 0.008 to 16.0 cm⁻¹ and 9.3 to 23.2 cm⁻¹, respectively. The reference phantom used for measurements made with the original system had a wavelength-averaged μₐ=2.0 cm⁻¹ and μₛ′=10.6 cm⁻¹. The correlation coefficients for μₐ and μₛ′ were 0.9981 and 0.9588, respectively, for optical properties quantified with the modified system. An overall error of 6.0±5.6% was calculated for μₐ and 6.1±4.7% for μₛ′ for the modified system. For the purposes of comparison, the original system had overall errors of 5.8±5.1 and 3.0±3.1% for extracting μₐ and μₛ′, respectively.

Exemplary Embodiment B. To assess the performance of a second embodiment of the modified diffuse reflectance spectroscopy system of the presently disclosed subject matter for measuring tissue optical properties, a series of experiments were performed on homogeneous liquid phantoms with absorption and reduced scattering coefficients (μₐ and μₛ′) similar to those of human breast tissue in the 400-600 nm wavelength range (see Cheong, 1995). Water soluble hemoglobin (H0267; Sigma-Aldrich Co., St. Louis, Mo., United States of America) and 1-μm diameter polystyrene spheres (07310-15; Polysciences, Inc., Warrington, Pa., United States of America) were used as the absorber and scatterer, respectively. The phantoms were made in a 3.5 cm diameter container and filled up to a height of at least 4 cm. A spectrophotometer (Cary 300; Varian, Palo Alto, Calif., United States of America) was used to measure the wavelength-dependent absorption coefficients of the stock hemoglobin solution used to create the phantoms. Prahl's Mie scattering program was used to determine the reduced scattering coefficient (Prahl, 2005).

Two sets of liquid phantoms were created and measured. The first set (S1) consisted of seven phantoms of different concentrations (3.7-34.9 μM) of the absorber and a fixed low number for scattering. The second set (S2) consisted of another seven phantoms of the same variable concentrations of Hb as S1, but with a fixed high number for scattering. The low and high scattering phantoms had a wavelength averaged μₛ′ of 10-14 cm⁻¹ and 16-23 cm⁻¹ over 400-600 nm, respectively. A summary of the optical properties of the phantom sets are provided in Table 1.

TABLE 1
Average Optical Properties over 400-
600 nm for Two Sets of Phantoms¹

| | | |
|---|---|---|
| S1 | S2 | S1 & S2 |

| Phantom | μₐ | μₛ′ | μₐ | μₛ′ | Hb (μM) |
|---|---|---|---|---|---|
| A | 0.8 | 13.6 | 0.8 | 23.1 | 3.7 |
| B | 1.7 | 13.1 | 1.7 | 22.2 | 7.9 |
| C | 2.5 | 12.6 | 2.5 | 21.4 | 11.6 |
| D | 3.8 | 11.9 | 3.8 | 20.1 | 17.5 |
| E | 5.0 | 11.2 | 5.0 | 18.9 | 23.3 |
| F | 6.3 | 10.4 | 6.3 | 17.7 | 29.1 |
| G | 7.5 | 9.7 | 7.5 | 16.4 | 34.9 |

¹μₐ and μₛ′ in cm⁻¹; Hb in μM.

LABVIEW™ software (National Instruments, Austin, Tex., United States of America) was used to control the monochromator, tuning the light source from 400-600 nm, and to digitally record diffuse reflectance measurements from the current amplifier. Prior to making optical measurements, the slit widths of the monochromator were optimized such that the output power from the illuminating fiber is maximized while the full-width at half-maximum (FWHM) of the lamp spectrum is 4.5 nm (to resolve the structure of the hemoglobin absorption bands). In the 400-600 nm range, the maximum power was 150 μW at 465 nm, and the minimum power was 50 μW at 600 nm. After a warm up time of 25 minutes, diffuse reflectance spectra were measured over the 400-600 nm wavelength range at increments of 5 nm. The measurements were repeated three times for each phantom to ensure good repeatability. The measurements were made with the room light off and the probe tip in contact with the surface of the liquid phantom. A measurement was also taken from a SPECTRALON® 99% diffuse reflectance standard (SRS-99-010; Labsphere, Inc., North Sutton, N.H., United States of America) with the probe tip in contact with the puck at the end of each phantom study. This spectrum was used to correct for the wavelength-dependent response of the system and throughput of the instrument. For the most absorbing phantom (S2-G) measured, the calculated average signal to noise ratio (SNR) over all wavelengths was 60±10 dB, with a minimum SNR of 41 dB at 400 nm and a maximum SNR of 84 dB at 480 nm. SNRλ was defined as

$$20 * log ( \frac{ I avg , λ}{σλ} )$$

where l is the intensity and σ is the standard deviation at the intensity, obtained from the three repeated measurements.

### Example 3

### Monte Carlo Model of Reflectance

An inverse Monte Carlo model of reflectance based on a scaling approach was used to extract μₐ and μₛ′ of the liquid phantoms. Extensive description of the model theory (see Palmer & Ramanujam, 2006a; Palmer & Ramanujam, 2006b; U.S. Patent Application Publication Nos. 2007/0232932 and 2008/0270091) and optimization of the algorithm for the extraction of biological absorption and scattering properties is briefly described hereinbelow.

The diffuse reflectance spectrum was a function of the wavelength dependent absorption and scattering coefficients, determined using the Beer-Lambert law and Mie theory, respectively. In the forward model, the diffuse reflectance spectra for a given range of absorption and scattering coefficients were generated by scaling a single baseline Monte Carlo simulation for a wide range of optical properties, which were then stored in a lookup table. The main assumptions for the model were that the absorbers present in the medium were known and that the scatterers were uniformly distributed single-sized spheres. Hemoglobin was the only absorber, and polystyrene spheres were the only scatterers in this case.

In the inverse model, the measured diffuse reflectance spectrum was fitted to the modeled diffuse reflectance spectrum by iteratively updating the free parameters, which included the hemoglobin concentration and the scatterer size and volume density. In the phantom studies, the fixed parameters were the extinction coefficients of the absorber and the wavelength-dependent refractive indices of the scatterer and surrounding medium, which are 1.6 and 1.33, respectively. When the sum of squares error of the modeled and measured spectra was minimized, the optical properties obtained from the extinction coefficients of the absorber and the wavelength-dependent refractive indices that best predict the measured diffuse reflectance spectrum were extracted.

The probe geometry was modeled by taking a microscopic image of the probe tip and digitally tracing the illumination fiber and the photodiode edges. The image was converted to a binary image that clearly delineated the illumination and detection areas of the probe. The scalable inverse Monte Carlo model was able to account for very specific probe geometries by convolving the photon collection probability over each source-detector point on the probe.

One parameter of probe geometry that the model took into account was the NA of the illumination and detection fibers. Since the detection fiber was replaced by a silicon photodiode, which has no nominal NA, the photodiode NA was experimentally obtained to feed into the MC model as the collection fiber NA. A laser diode was collimated to excite the active area of the photodiode, which was mounted on a rotation stage. With no ambient light in the room, a current amplifier was used to monitor the signal due to the laser while rotating the photodiode to determine the maximum acceptance angle. A measured acceptance angle of 75° in air gave an NA of 0.965 for the photodiode.

To calibrate for system throughput and wavelength dependence, the experimentally measured and modeled spectra of the target phantom were normalized to that of a reference phantom with predefined optical properties at each wavelength. Phantom B in phantom set 2 (a low-absorbing phantom with μₐ=1.7 cm⁻¹ and μₛ′=22.2 cm⁻¹) was used as the reference phantom to calibrate every other phantom as targets within each phantom set. The reference phantoms were chosen based on a comprehensive study on the robustness of the inverse MC model in extracting a wide range of optical properties. Optical properties at each wavelength were extracted for each target phantom, and the inversion errors were averaged over all wavelengths and phantoms. The inversion errors were evaluated based on the following criteria. Extracted errors of less than 10% were considered excellent while errors of 10-20% were good. Errors above 20% in phantoms were considered high and might not accurately extract physiological parameters in tissue.

### Example 4

### Simulation of Wavelength Reduction

The potential for replacing the Xenon lamp and monochromator with one or more LEDs in the 400-600 nm range was investigated by performing simulations of wavelength reduction on the measured liquid phantom data obtained with the presently disclosed modified system. Five (5) commercially available LED wavelengths in the 400-600 nm spectral range were chosen: 405, 450, 470, 530, and 590 nm.

An assumption in the simulation was that each wavelength has a bandwidth of 20 nm with a Gaussian distribution. This was an approximation made based on the commercially available LED specifications. The collected spectra from the phantom studies were processed such that data points from all wavelengths were excluded, except for those of the LED wavelengths enumerated previously. Each originally measured phantom spectrum, which included 41 wavelengths over the 400-600 nm range in 5 nm increments, was first convolved with each of the five (5) Gaussian-distributed LED emission spectra separately. This generated five (5) individual new spectra. Then the new spectra were integrated over 100 nm, an arbitrarily large value that spans much wider than the LED bandwidth of 20 nm, to account for all potential signals from the LEDs. The integration was desirable because with a single photodiode, only the integrated intensity of the new spectrum can be measured. The resulting five (5) intensities were the signals that would be measured using those specific LEDs. The final wavelength-reduced spectrum for each of the phantoms was composed of only these five (5) data points. These newly generated LED spectra were used to extract optical properties.

### Example 5

### Simulated Crosstalk Analysis

The single-pixel device (e.g., a device having an optical probe with a tip like those depicted in FIGS. 2B and 2C) disclosed herein can be multiplexed into a quantitative spectral imaging device. This can be accomplished by arranging multiple optical fiber-photodiode pairs in a matrix formation. A parameter that can be characterized is the crosstalk. In an ideal situation, a fiber-photodiode pair can be treated as a single pixel; however, the issue of a detector collecting stray light from an adjacent pixel, or even from multiple adjacent pixels, can also be considered. High levels of crosstalk can affect the measurement accuracy from tissue directly below the pixel.

To demonstrate feasibility of implementing a quantitative spectral imaging device, a Monte Carlo forward model of reflectance as described hereinabove was used to simulate a design where nine (9) Hamamatsu S1227-66BR photodiodes, each with 1.3 mm holes drilled in the center, were packed as closely together in a 3×3 matrix as shown in FIG. 5. Each fiber was 1 mm in diameter and had an NA of 0.22. The silicon photodiode NA was 0.965. The separation of adjacent fibers was 8.48 mm. A forward model based on this geometry was used to generate the diffuse reflectance spectrum including both signal and cross-talk for each pixel. The simulated spectrum from each pixel was then inverted independently to determine the effect of crosstalk on the extracted optical properties.

The extracted errors due to the presence cross-talk were estimated by simulating phantom measurements with hemoglobin as the absorber and polystyrene spheres as the scatterer. Measurements were simulated for five (5) phantoms with a wide range of average absorption coefficients over 400-600 nm (μₐ=0.4, 0.9, 1.3, 1.6, 2.0 cm⁻¹) and a fixed reduced scattering coefficient (μₛ′=10). The inversion accuracy in the presence of crosstalk not only provided feasibility of creating such a device, but also useful information for additional design parameters such as fiber size, detector size, and pixel spacing.

### Example 6

### Comparison of Prior Benchtop System with a Modified System

The benchtop system depicted generally in FIG. 1 was modified to decrease its size and cost while still achieving comparable performance in extracting tissue optical properties. The modification of the benchtop system not only impacted size and cost but also the ability to multiplex the device into a quantitative spectral imaging system. Comparisons of the throughput-related parameters and system characteristics of the original and modified systems are presented in Table 2.

TABLE 2
Comparison of Throughput-related Parameters
of Benchtop and Modified Systems

| | Original System | | Modified System | |
|---|---|---|---|---|
| Illumination Sources | Xenon lamp and | | Xenon lamp and | |
| | Monochromator | | Monochromator | |
| | (Reflectance and | | (Reflectance only) | |
| | Fluorescence) | | | |
| Effective | 1.00 | mm | 1.04 | mm |
| Illumination | | | | |
| Diameter | | | | |
| Illumination NA | 0.22 | | 0.22 | |
| Detection Areas | 2.26 | mm² | 32.31 | mm² |
| Detection NA | 0.22 | | 0.96 | |
| Sensing depth | 0.6-1.4 | mm | 0.4-1.7 | mm |

| (over 400-600 nm) | μₐ = 0.5~2.5 cm⁻¹, | μₐ = 0.5~2.5 cm⁻¹, | |
|---|---|---|---|
| | μₛ′ = 10~20 cm⁻¹) | μₛ′ = 10~20 cm⁻¹) | |
| Detector QE | 35% (400~600 nm) | 73% (400~600 nm) | |
| | Min: 26% @ 450 nm | Min: 62% @ 400 nm | |
| | Max: 45% @ 600 nm | Max: 79% @ 600 nm | |
| Dark Noise | 6.4 × 10⁻⁷ pA | 20 | pA |

| | | |
|---|---|---|
| Readout Noise | 4.2 × 10⁻⁹ A | 1 × 10⁻¹² A |
| SNR (400~600 nm) | Average: 45 ± 5 dB | Average: 60 ± 10 dB |
| μₐ = 7.5 cm⁻¹, | Min: 32 dB @ 405 nm | Min: 41 dB @ 400 nm |
| μₛ′ = 16 cm⁻¹) | Max: 60 dB @ 550 nm | Max: 84 dB @ 480 nm |
| Cost of Detection | >$20,000 | #1,000 |
| System | | |

Certain limitations of side-by-side comparisons of various parameters of the prior benchtop and the modified system of the presently disclosed subject matter were identified. In some embodiments, the modified system used a monochromator to tune the light from a Xenon lamp from 400-600 nm, which was directly illuminated onto the sample. On the other hand, the original system used only white light to illuminate the sample, and the collected light was then split by the spectrograph. The monochromator was used in this particular instance because it was readily available. Because the monochromator was relatively slow in scanning a range of wavelengths, taking over a minute for a measurement, in some embodiments a filter wheel can be implemented in the place of the monochromator to speed up data acquisition in systems designed to employ a tunable source. In some embodiments, the monochromator can be replaced by a filter wheel with multiple filter positions including, but not limited to 400, 420, 440, 470, 500, 530, 570, 600 nm.

Since the effective illumination diameter and source detector separation were similar for both systems, the sensing depth was also similar over the same range of wavelengths for a given set of optical properties. Monte Carlo simulations were performed to assess sensing depth for both probes over 400-600 nm for the optical properties, μₐ=0.5-2.5 cm⁻¹ and μₛ′=10-20 cm⁻¹. The sensing depth was defined as the depth at which 90% of the probable visited photons in the sample exited and reached the detector to be collected. The modified system had a slightly deeper sensing depth because the detection area was bigger and could collect photons that had traveled deeper into the medium although these exit photons farther away from the illumination fiber had much less weight than those that were closer to the illumination fiber. The sensing depth can be easily altered by adjusting various source-detector separations and is a parameter that can be considered in alternative probe designs, for example depending on the clinical application for which the technology is to be used.

While some parameters, such as sensing depth and effective illumination area, were comparable for both systems, the modified system had several parameters that were superior to those of the original system, which ultimately translated to a higher signal-to-noise ratio (SNR), and lower cost. Based on the commercial specification sheets, the CCD of the benchtop system had an average quantum efficiency of 35% from 400-600 nm. On the other hand, the photodiode in the modified system had an average quantum efficiency of 73% in the same range. Furthermore, the detector was directly in contact with the sample in the modified design, collecting most of the remitted light, whereas the detector of the benchtop system was at the distal end of the collection fiber bundle where significant light can be lost. The average SNR in a dark, highly absorbing phantom (μₐ=7.5 cm⁻¹ and μₛ′=16 cm⁻¹) measured using benchtop system was 45±5 dB over 400-600 nm, which was lower than the 60±10 dB measured with the modified system. In addition, the cost of the detection portion of the modified system was considerably less than that of its benchtop counterpart.

### Example 7

### Synthetic Tissue Phantom Studies

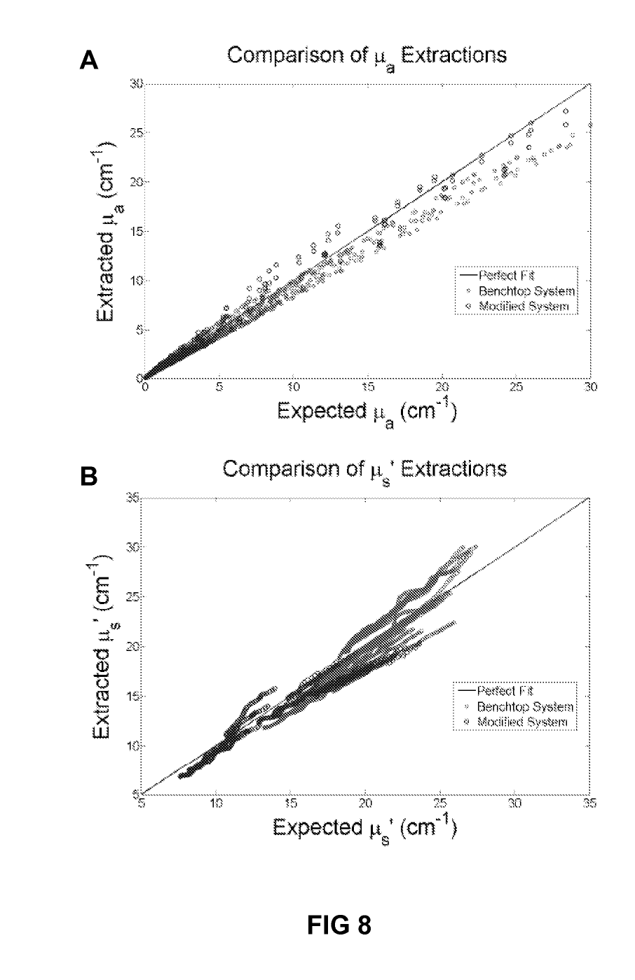

Monte Carlo inversions were performed to extract optical properties on both sets of phantoms. FIGS. 8A and 8B show the extraction performance using the modified system of the presently disclosed subject matter along side the prior benchtop system. For the modified system, the correlation coefficients for expected and extracted μₐ and μₛ′ were 0.9992 and 0.9478, respectively. Using phantom S2-B (μₐ=1.7 cm⁻¹ and μₛ′=22.2 cm⁻¹) as the reference, the overall extracted μₐ error was 9.8±5.0%, and the overall μₛ′ error was 7.6±4.2%. For this similarly wide range of optical properties and using a similar reference phantom (μₐ=1.4 cm⁻¹; μₛ′=19.3 cm⁻¹), the original benchtop system had overall errors of 9.8±8.2% and 7.7±6.3% for μₐ and μₛ′, respectively. All percent error values given were mean RMS percent errors averaged across all wavelengths for all target phantoms for the extraction of optical properties. The modified system of the presently disclosed subject matter and the prior benchtop system thus had very comparable performance in extracting optical properties in tissue phantoms over a wide range of optical properties.

### Example 8

### Simulated Wavelength Reduction

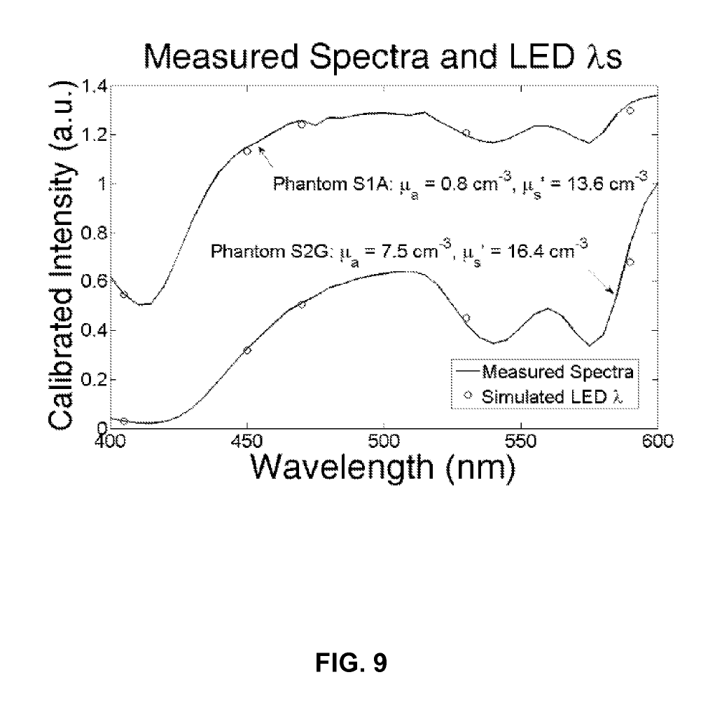

FIG. 9 shows the measured reflectance spectra of the lowest and highest absorbing phantoms for all wavelengths and the generated data points from the wavelength reduction simulation used for additional MC inversions, both calibrated by the puck spectrum. The simulated wavelength-reduced spectra were composed on only five data points each. These five data points were the signal that would be read by the photodiode current amplifier.

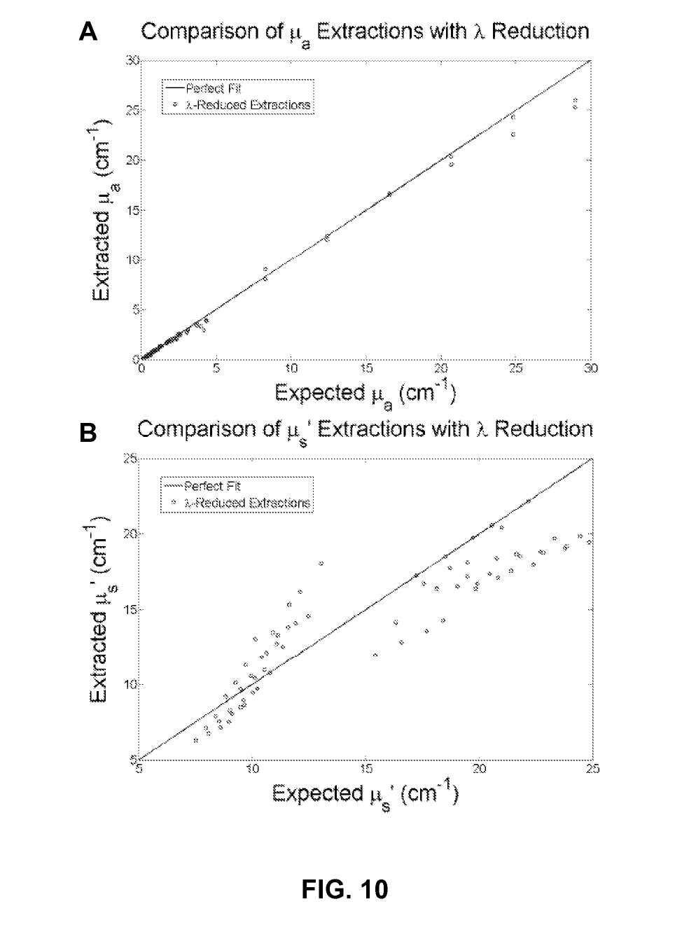

FIGS. 10A and 10B illustrate the theoretical extraction performance of the modified system of the presently disclosed subject matter after wavelength reduction simulations. For the same large range of optical properties and using the same reference phantom as before (S1-B: μₐ=1.7 cm⁻¹ and μₛ′=22.2 cm⁻¹), the overall μₐ extraction error was 9.6±5.8%, and the overall μₛ′ error was 14.3±7.3%. The correlation coefficients for expected and extracted μₐ and μₛ′ were 0.9972 and 0.8628, respectively, in the inversion of wavelength-reduced phantom data. The increase in the extraction errors can be attributed to not only the reduction of wavelengths, but also the loss of spectral information with a wider FWHM (20 nm) of the simulated wavelength reduction.

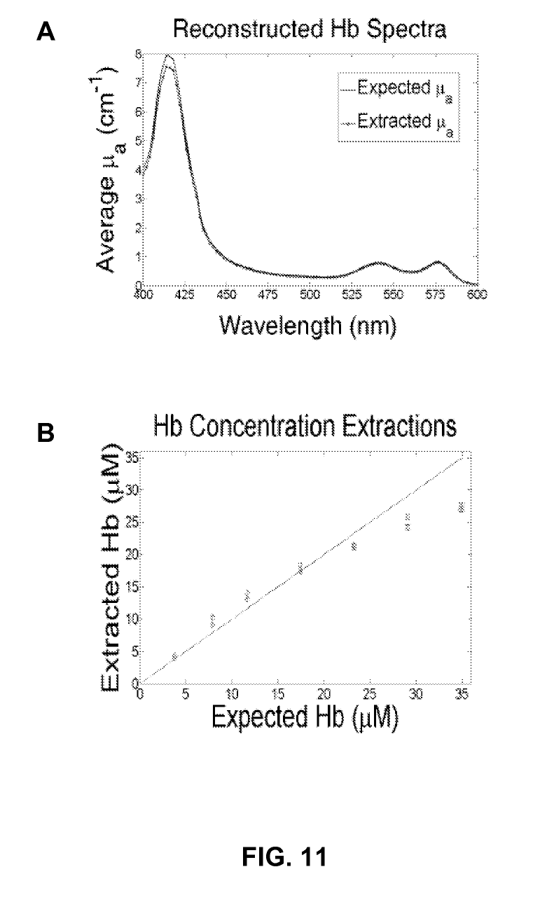

Using only five wavelengths from the collected phantom data to perform the Monte Carlo inversion, the hemoglobin spectra was reconstructed with the extracted absorption coefficients and the molar extinction coefficient for hemoglobin measured with the spectrophotometer on the day of the phantom study. FIG. 11A shows the reconstructed hemoglobin spectra averaged over all phantoms. FIG. 11B shows relatively good extraction accuracy for hemoglobin concentrations for all phantoms. There was a slight underestimation of hemoglobin at very high concentrations, which was consistent with previous studies using the prior benchtop system.

These wavelength reduction results showed the feasibility of replacing the Xenon Arc lamp and the monochromator in the modified system with just five LEDs in some embodiments of the presently disclosed subject matter. Not only is there an abundance of high-powered LEDs in the 400-600 nm range, these potential light sources are also very inexpensive. Furthermore, the use of LEDs can potentially obviate the need for optical fibers and is well-suited for miniaturized optical spectral imaging systems. With LEDs as the illumination source and tiny photodiodes as the detector (see e.g., FIGS. 3A and 3C), the device would be considerably smaller than the prior benchtop system while still achieving comparable performance in the extraction of optical properties in tissue. Additionally, an LED-photodiode device would be expected to have not only the benefits of having the superior collection efficiency of the detector, but also higher throughput with high-powered LEDs.

In addition to LEDs as an alternative source, a combination of a lamp and a series of band-pass filters can also be implemented. The use of band-pass filters in conjunction with an optical fiber can also provide high throughput similar to LEDs and is relatively simple to integrate into the benchtop system. However, a potential disadvantage of using the latter approach would be the increased cost and size of a lamp-filter wheel based system. The enumerated errors of the extraction of optical properties shown in Table 3 indicated that it was unnecessary to use the full 400-600 spectrum to extract optical properties with good accuracy.

TABLE 3
Comparison of the Benchtop System and the Modified System
with its Wavelength-reduced Inversion Errors, Averaged
for all Reference-target Phantom Combinations
Summary of Optical Properties and Inversion Errors

| | Avg μₐ | Avg μₛ′ | | | |
|---|---|---|---|---|---|
| (400-600 | Range | Range | Hb Range | μₐ Error | μₛ′ Error |
| nm) | (cm⁻¹) | (cm⁻¹) | (μM) | (%) | (%) |
| Benchtop | 0.2~82 | 16.9~24.1 | 1.0~35.2 | 9.8 ± 8.2 | 7.7 ± 6.3 |
| System | | | | | |
| Modified | 0.8~7.5 | 9.7~23.1 | 3.7~34.9 | 9.8 ± 5.0 | 7.6 ± 4.2 |
| System | | | | | |
| λ-Reduced | 0.8~7.5 | 9.7~23.1 | 3.7~34.9 | 9.6 ± 5.8 | 14.3 ± 7.3 |

Wavelength choice can be relevant when the system is used in clinical situations. The phantoms presented herein were simplified as compared to the composition of real human tissue. However, it is recognized from several studies that hemoglobin is the dominant absorber in tissue. Its concentration can be extracted with good accuracy with a few wavelengths using the presently disclosed subject matter. The current wavelength choices presented herein sufficiently encompass the distinct features of hemoglobin: the Soret, α-, and β-bands. Oxy- and deoxy-hemoglobin and thus hemoglobin saturation can be extracted because of the clear shifts in spectral peaks. These are relevant parameters that can be used to delineate normal from malignant tissues. Other physiological parameters can also be quantified using just a few wavelengths, analogous to other systems currently in clinical studies, such as those using frequency domain photon-migration techniques (Fishkin et al., 1997). If more than 5 wavelengths are needed to accurately extract other important physiological parameters, a system with a lamp and filter wheel can be designed to accommodate as many as 10 wavelengths. The addition of a few extra LEDs can also be implemented.

### Example 9

### Simulated Crosstalk Analysis

Crosstalk was also simulated. It was hypothesized that the center pixel in 3×3 matrix, shown previously in FIG. 7, would receive the most amount of crosstalk and thus was presented as a worst case scenario. As expected, the inversion showed that the center detector had the worst extraction errors for μₐ and μₛ′. Table 4 presents the inversion errors in the presence of crosstalk at the center, the side, and the corner detectors, respectively.

TABLE 4
Extraction Errors (%) for Each Detector in the Presence of Crosstalk²
Inversion Errors with Crosstalk

| | Center Detector | | Side Detectors | | Corner Detectors | |
|---|---|---|---|---|---|---|
| Phantoms | μₐ error | μₛ′ error | μₐ error | μₛ′ error | μₐ error | μₛ′ error |
| A | 2.2 | 7.8 | 1.6 | 5.7 | 1.0 | 2.9 |
| B | 2.2 | 5.1 | 1.6 | 3.6 | 0.9 | 1.8 |
| C | 2.4 | 5.0 | 1.6 | 3.3 | 0.9 | 1.8 |
| D | 3.6 | 6.5 | 1.8 | 3.8 | 1.3 | 2.3 |
| E | 4.3 | 8.1 | 2.4 | 4.8 | 1.7 | 3.1 |

²in phantoms ranging from low to high absorption coefficients (μₐ = 0.4-2.0 cm⁻¹) and mid reduced scattering coefficients (μₛ′ = 10 cm⁻¹), averaged for all reference-target phantom combinations.

The errors were averaged over all reference-target phantom combinations. With μₐ and μₛ′ extraction errors of less than 2% and 5%, respectively, the simulation showed that crosstalk had little effect on the side and corner detectors. The center detector received the most crosstalk, and its extraction errors were nearly double those of the non-center detectors. Simulation showed that the overall errors due to crosstalk were relatively small and that constructing an imaging device is feasible based on this particular geometry. Other factors that could reduce crosstalk errors in the multi-pixel device prototype include, but are not limited to fiber size, detector size, and detector spacing.

### Discussion of the Examples

Disclosed herein are optical probes, systems, and methods that use a multimode fiber coupled to a tunable light source for illumination and a photodiode (e.g., a 2.4-mm photodiode) for detection in contact with a tissue surface. The presently disclosed optical probes coupled with an inverse Monte Carlo model of reflectance is demonstrated to accurately quantify tissue absorption and scattering in tissue-like turbid synthetic phantoms with a wide range of optical properties. The overall errors for quantifying the absorption and scattering coefficients were 6.0±5.6 and 6.1±4.7%, respectively. Compared to fiber-based detection, having the detector right at the tissue surface can significantly improve light collection efficiency, thus reducing the requirement for sophisticated detectors with high sensitivity. This disclosed optical probes can be easily expanded into a quantitative spectral imaging system for mapping tissue optical properties in vivo.

The modified system disclosed herein can be used to quantified absorption from phantoms with absorption coefficients up to at least 10 cm⁻¹. Compared to the prior system, the modified system of the presently disclosed subject matter had slightly higher errors in extraction of scattering coefficient, presumably due to its 10 to 15-dB lower SNR for high scattering. The dynamic range of the disclosed system can be improved by decreasing the center-to-center distance between the source and detector and/or by increasing the area of the photodiode.

The modified system combined with the MC model employed can be extended into an optical spectral imaging system to map out the concentrations of absorbers and the bulk tissue scattering properties of subsurface tissue volumes, which are on a length scale of several millimeters. There are many applications for which the presently disclosed subject matter can be employed, including, but not limited to epithelial pre-cancer and cancer detection (such as but not limited to those of the skin, oral cavity, and cervix), intraoperative tumor margin assessment, and the monitoring of tumor response to therapy in organ sites such but not limited to the head and neck and cervix. Additionally, the ability of the presently disclosed optical probes to be placed directly at the tissue surface can improve collection efficiency and can eliminate the need to use expensive CCDs.

Additionally, wavelength reduction simulations were also performed to assess the feasibility of replacing the tunable light source with several miniature LEDs. Crosstalk analyses indicated that the system can be multiplexed into an imaging device, which can be employed to quantify tissue physiological and morphological properties over a large field of view.

This multi-faceted study shows that the modified system along with our Monte Carlo model can be miniaturized and extended into an optical spectral imaging system. In its current, single-pixel state, the system is capable of extracting optical properties in tissue phantoms with good accuracy in the 400-600 nm range comparable to the clinical benchtop system, and accuracy out to 950 nm is also expected. By placing the detector directly in contact with the sample, the collection efficiency is improved. Furthermore, the results from the wavelength reduction simulations from the measured phantom data show great potential in replacing the lamp and monochromator with several high powered LEDs in the 400-600 nm range for higher throughput, smaller size, and much lower cost. By strategically choosing high powered LEDs with a 20-30 nm bandwidth while covering most of the 400-600 nm range, an LED-photodiode device can be created and used to extract a similar range of tissue optical properties within a well-defined sensing depth. The new semiconductor device would not only undoubtedly have higher throughput than the lamp-monochromator model, but also be truly miniaturized and made at a fraction of the cost of the original system. Lastly, the crosstalk analysis shows the potential for either the fiber-photodiode system or the miniaturized LED-photodiode system to be multiplexed into an imaging device. With a low probability of exiting photons reaching adjacent detectors, the effect of crosstalk on inversion accuracy is low for a matrix of 5.8×5.8 mm silicon photodiodes. By accurately accounting for crosstalk with our Monte Carlo model, an imaging system can be made with much smaller detectors spaced closer to one another. The use of smaller, more sensitive detectors along with sources with superior throughput is an aspect of the presently disclosed subject matter.

The eventual goal of creating a miniaturized spectral imaging device based on inexpensive photodiodes and LEDs can have a remarkable impact in not only basic biomedical research, but also in clinical situations worldwide. While a single-pixel probe is certainly useful for small regions of tissue, the information that can be unraveled by an imaging device is unmatched for larger samples, such as those in tumor margin assessment, assessing tumor response to therapy, epithelial pre-cancer and cancer detection, among other applications. A miniaturized imaging device based on the LED-photodiode design can spectrally map out quantitative biological information for tissue composition just below the surface. Furthermore, the device is portable and inexpensive, useful and accessible for not only the standard research laboratory or clinic, but also for rural clinics in the developing world.

### REFERENCES

All references listed below, as well as all references cited in the instant disclosure, including but not limited to all patents, patent applications and publications thereof, and scientific journal articles, are incorporated herein by reference in their entireties to the extent not inconsistent herewith and to the extent that they supplement, explain, provide a background for, and/or teach methodology, techniques, and/or compositions employed herein.
Bigio & Bown (2004) Cancer Biother 3:259-267.
Bohren & Huffman (1983) Absorption and Scattering of Light by Small Particles, John Wiley & Sons, Inc., New York, N.Y., United States of America.
Cerussi et al. (2006) J Biomed Opt 11:044005.
Cheong (1995) in Optical-Thermal Response of Laser-Irradiated Tissue, Lasers, Photonics, and Electro-optics. Welch & Gemert (eds), Plenum Press, New York, N.Y., United States of America, pp. 275-303.
Feather et al. (1988) Phys Med Biol 33:711-722.
Fish kin et al. (1997) Appl Opt 36:10-20.
Huffman (1998) Absorption and Scattering of Light by Small Particles, John Wiley & Sons, Inc., New York, N.Y., United States of America.
Lin et al. (2001) Photochem Photobiol 73:396-402.
Lo et al. (2009) Optics Express 17:1372-1384.
Mirabal et al. (2002) J Biomed Opt 7:587-594.
Palmer & Ramanujam (2006a) Appl Opt 45:1062-1071.
Palmer & Ramanujam (2006b) Appl Opt 45:1072-1078.
PCT International Patent Application Serial Nos. PCT/US2006/028770, PCT/US2007/006624, PCT/US2007/007586, PCT/US2008/002431, PCT/US2008/078186, PCT/US2008/078194.
U.S. Patent Application Publication Nos. 2003/0220549; 2004/0162489; 2005/0143663; 2005/0203419; 2006/0247532; 2007/0019199; 2007/0201788; 2007/0232932; 2008/0270091; 2009/0015826.
Yu et al. (2008) J Biomed Opt 13:060505.
Zhu et al. (2005) J Biomed Opt 10:024032.
Zhu et al. (2006) Lasers Surg Med 38:714-724.
Zonios et al. (1999) Appl Opt 38:6628-6637.

It will be understood that various details of the presently disclosed subject matter may be changed without departing from the scope of the presently disclosed subject matter. Furthermore, the foregoing description is for the purpose of illustration only, and not for the purpose of limitation.

1. A diffuse reflectance spectroscopy system for quantifying light absorption and scattering in a tissue mass, the system comprising:
an optical probe comprising at least one entity for emitting light that interacts with a tissue mass and then is remitted into a collecting entity, wherein the collecting entity comprises a detector comprising one or more photodiodes; and
a processing unit for converting collected light, via a Monte Carlo algorithm or a diffusion algorithm into absorption and scattering data.

2. The diffuse reflectance spectroscopy system of claim 1, wherein the entity for emitting light is present at a fixed distance external to a photodiode.

3. The diffuse reflectance spectroscopy system of claim 1, wherein the entity for emitting light comprises one or more illumination fibers, each illumination fiber being present at a fixed distance external to a photodiode, optionally adjacent to a photodiode.

4. The diffuse reflectance spectroscopy system of claim 1, wherein the entity for emitting light comprises one or more illumination fibers, each illumination fiber being present within a photodiode.

5. (canceled)

6. The diffuse reflectance spectroscopy system of claim 4, wherein the photodiode comprises an aperture, and the illumination fiber is disposed within the aperture, optionally wherein spacing is present to vary the distance between the center of the aperture and/or fiber and an edge of the photodiode.

7. The diffuse reflectance spectroscopy system of claim 1, further comprising a light source coupled to the entity for emitting light, wherein the light source optionally comprises a lamp or a plurality of light-emitting diodes (LEDs).

8-9. (canceled)

10. The diffuse reflectance spectroscopy system of claim 1, wherein the entity for emitting light comprises direct illumination via a lamp or a plurality of light-emitting diodes (LEDs).

11-12. (canceled)

13. The diffuse reflectance spectroscopy system of claim 10, wherein the entity for emitting light and collecting entities are encased in a housing, where the entity for emitting light is at a proximal end of the housing and the one or more photodiodes are at a distal end of the housing, the one or more photodiodes each comprising an aperture, whereby the entity for emitting light provides backlit illumination through each aperture into one or more photodiodes.

14. The diffuse reflectance spectroscopy system of claim 13, wherein the housing comprises one or more reflective interior surfaces.

15. The diffuse reflectance spectroscopy system of claim 1, wherein the one or more photodiodes comprises an array of photodiodes.

16-17. (canceled)

18. An optical probe comprising at least one entity for emitting light into a tissue mass and at least one collecting entity for collecting light that has interacted with a tissue mass, wherein the collecting entity comprises one or more photodiodes.

19. The optical probe of claim 18, wherein the entity for emitting light is present at a fixed distance external to a photodiode.

20. The optical probe of claim 19, wherein the entity for emitting light comprises one or more illumination fibers, each illumination fiber being present at a fixed distance external to a photodiode.

21. The optical probe of claim 18, wherein the entity for emitting light comprises one or more LEDs.

22. (canceled)

23. The optical probe of claim 18, wherein the optical probe further comprises a housing, and the entity for emitting light is at a proximal end of the housing and the one or more photodiodes are at a distal end of the housing, whereby the entity for emitting light provides backlit electromagnetic radiation with respect to the one or more photodiodes.

24. The optical probe of claim 23, wherein the housing comprises one or more reflective interior surfaces.

25. The optical probe of claim 18, comprising one or more illumination fibers, each illumination fiber being present within a photodiode.

26. The optical probe of claim 25, wherein the illumination fiber is disposed longitudinally along the center of the photodiode.

27. The optical probe of claim 25, comprising a buffer between the photodiode and the illumination fiber.

28. The optical probe of claim 18, wherein the one or more photodiodes comprises an array of photodiodes.

29-50. (canceled)
